(19) United States
(12) Patent Application Publication
(10) Pub. No.: US 2025/0319047 A1
(43) Pub. Date: Oct. 16, 2025

(54) ANTIMICROBIAL TREATMENT AND PREVENTION BY PERILLYL ALCOHOL AND DERIVATIVES

(71) Applicants: Jose Zayas Rivera, Raleigh, NC (US); Lourdes M. Ramos de Velasco, Raleigh, NC (US)

(72) Inventors: Jose Zayas Rivera, Raleigh, NC (US); Lourdes M. Ramos de Velasco, Raleigh, NC (US)

(21) Appl. No.: 18/633,441

(22) Filed: Apr. 11, 2024

(51) Int. Cl. A61K 31/15; A61K 8/40; A61K 9/00; A61K 9/06; A61K 31/045; A61K 31/167; A61K 47/10; A61K 47/12; A61K 47/22; A61K 47/26; A61K 47/32; A61K 47/38; A61K 47/40; A61K 47/44; A61P 31/04; A61P 31/10; A61Q 11/00

(52) U.S. Cl. CPC A61K 31/15; A61K 8/40; A61K 9/0056; A61K 9/06; A61K 31/045; A61K 31/167; A61K 47/10; A61K 47/12; A61K 47/22; A61K 47/26; A61K 47/32; A61K 47/38; A61K 47/40; A61K 47/44; A61P 31/04; A61P 31/10; A61Q 11/00

(57) ABSTRACT

A formulation including perillyl alcohol and/or derivatives is provided, and a method to inhibit the growth of bacteria, fungi, and yeasts in the presence of blood, bodily fluids, and soil is demonstrated. The use of perillyl alcohol and derivatives, pure or in formulations, is used to inhibit the growth of bacteria, fungi, and yeasts in the presence of blood, bodily fluids, and soil. The formulation is administered to a subject in need thereof an effective amount of a formulation comprising at least one monoterpene as an active ingredient in a concentration between 0.1% to 3%, wherein the formulation remains active in the presence of blood, inorganic material, organic material, and bodily fluids.

### FIELD OF THE INVENTION

The invention relates to the capacity of perillyl alcohol, and derivatives to inhibit the growth of bacteria, fungi, and yeasts in the presence of blood, bodily fluids, and soil. Specifically, the invention relates to the use of perillyl alcohol and derivatives, pure or in formulations, to inhibit the growth of bacteria, fungi, and yeasts in the presence of blood, bodily fluids, and soil.

### BACKGROUND OF THE INVENTION

A disinfectant or antiseptic is defined as a substance with the ability to reduce the microbial load on a surface. Mainly those microorganisms that can cause negative effects, and when related to humans or animals, are known as pathogenic and can originate infections. When related to skin, it is the action exerted by the antimicrobial over the upper or outer layer of the skin as illustrated in FIG. 1.

The treatment for skin disinfection is currently approached using several types or classes of disinfectants (Table 1). Among them are biguanides (e.g., chlorhexidine), and iodine (e.g., Povidone-Iodine, PI), which are the most frequently used. They have been shown to be effective against a plethora of pathogenic microorganisms. The mechanism of action for chlorhexidine gluconate (CHG) is believed to be via two pathways (Lim, Anaesthesia and Intensive Care, Vol. 36, No. 4, July 2008). At bacteriostatic or low concentration, CHG disrupts the bacterial cytoplasmic membrane causing leakage, whereas at high concentration it is bactericidal by penetrating the cytoplasm through the damaged cytoplasmic membrane.

TABLE 1

| Skin Disinfectant | Mechanism of Action |
|---|---|
| Chlorhexidine Gluconate | Disrupts microbial cell membranes |
| Povidone-Iodine | Oxidizes lipids of the cell membrane and |
| | forms salts with microbial proteins |
| Chloroxylenol | Disrupts microbial cell membranes |
| Isopropyl alcohol | Cell membrane damage and denatures |
| | proteins |
| Hexachlorophene | Disrupts microbial cell membranes |
| Benzalkonium Chloride | Disrupts microbial cell membranes |
| Hydrogen Peroxide | Oxidizes essential cell components |

Aside from the proposed mechanism of action, they all are limited to the disinfection of the epidermis. They are all negatively affected by the presence of organic matter, and in the case of CHG and PI, they are extremely sensitive to organic soil and are also affected by hard water. Additionally, they are not to be used on or near mucous membranes (Fuller J K, Fuller J R. Surgical Technology: Principles and Practice. “Surgical Skin Prep and Draping.” Elsevier Health Sciences; 2013) or cartilaginous tissue (Daeschlein G. Antimicrobial and antiseptic strategies in wound management. Int Wound J 2013; (suppl. 1): 9-14). Furthermore, research has shown that CHG loses effectiveness in the presence of organic matter, bodily fluids, blood, and soap. CHG is toxic and should not be used near mucous membranes (including cornea and ear). Furthermore, its primary degradation product (p-chloroaniline) is hematotoxic, nephrotoxic, hepatotoxic, and rapidly absorbed and metabolized. A recent study conducted by CADTH, an independent, non-profit organization, found that there seems to be no difference between CHG and povidone-iodine solutions containing high levels of alcohol. Recorded adverse events, such as fires in the Operating Room (OR), severe allergic reactions, anaphylactic shocks, and skin chemical burns, caused by CHG have only increased. Similarly, skin adverse events related to povidone-iodine (PI) have been reported, with no statistically significant difference in the incidence rate compared to CHG. Recent studies have shown that excessive use of CHG for multiple applications in disinfection throughout the decades has caused reduced efficacy of the agent against multidrug-resistant (MDR) bacteria such as K. pneumoniae and the overexposure associated with the emergence of resistance to Colistin. In some cases, the overuse of CHG is believed to be causing bacteria, such as A. baumannii, to become resistant to other antibiotics.

U.S. Pat. No. 5,994,598 A to Chastain et al., discloses that perillyl alcohol is an antimicrobial monoterpene derived from the lavender plant or from the oxidation of limonene (U.S. Pat. No. 5,574,195 A). In the patent, Chastain prepares different formulations of the monoterpene, including liquid, dentifrice, gel, paste, ointment and suppositories, paint, water-in-oil emulsion, soaps, and creams among others.

More recently, U.S. Pat. No. 9,271,492 B2 to Cornmell et al., described a composition for the use of antimicrobials and method for surface disinfection using selected monoterpenes. The recommendations include a detailed list of solvents and excipients that can be used to formulate the monoterpenes to be used for disinfection, including the skin surface.

The drawback to the prior art is that the disinfection process proposed is limited to the intact surface or epidermis in humans and animals. The formulations do not target other layers of the skin and leave wounds exposed to pathogenic microorganisms and do not address the main purpose of disinfection. That is which is to reduce the microorganisms that can create wound infection, with the subsequent issues related to sepsis, amputation of limbs, and death. This is greatly compounded in trauma centers and surgical applications through surgical site infections.

The process of skin disinfection covers temporary protection over the surface, i.e., epidermis. Once the epidermis is open or broken, the functionality of the disinfectant is reduced since there will be penetration of airborne microorganisms into the deeper layers like the dermis, and depending on depth of abrasion or cut it can reach the subcutaneous tissue or hypodermis as illustrated in FIG. 2. The skin can suffer this damage through dryness or incision as in a surgical procedure, cut, or other trauma. Once the epidermis or hypodermis is reached, capillaries and blood vessels will be lacerated, and the presence of blood and other fluids will deactivate the antiseptics spread over the skin. This is critical in the process of disinfection, as loss of the antiseptic's activity will render the cut or wound exposed to pathogenic microorganisms and thus infection.

### SUMMARY OF THE INVENTION

The invention provides perillyl alcohol, its derivatives, and other oxidized monoterpenes as an effective bactericidal and fungicidal agent in the presence of blood, bodily fluids, and soil in skin and open wounds in human and animals, as well as provide for critical prevention on the formation of biofilms.

According to an aspect of the invention, an active compound and its formulations is provided, together with other monoterpenes that will provide the necessary disinfection of the wound not only at the surface level, but at the open wound.

According to another aspect of the invention, a method for reducing pathogenic microorganisms on a subject is provided.

According to yet another aspect of the invention, a method of reducing pathogenic microorganisms on a subject comprises administering to a subject in need thereof an effective amount of a formulation comprising at least one monoterpene as an active ingredient.

According to still another aspect of the invention, the at least one monoterpene is selected from the group consisting of Perillyl alcohol, Perillaldehyde, Perillyl ester, Perillyl Imine, Perillartine, and combinations thereof.

According to an aspect of the invention, the at least one monoterpene is provided in a concentration between 0.1% to 3%.

According to another aspect of the invention, the formulation remains active in the presence of blood, inorganic material, organic material, and bodily fluids.

In accordance to still another aspect of the invention, the formulation remains active at least for 24 hours.

According to yet another aspect of the invention, the formulation is administered topically on a skin region of said subject and the skin region can also include a wound.

According to another aspect of the invention, the formulation remains active when the wound is occluded.

According to one aspect of the invention, the formulation is in the form of a gel, cream, ointment, or foam.

According to still another aspect of the invention, the pathogenic microorganisms comprise at least one of Gram (−) microorganisms, Gram (+) microorganisms, yeast, or fungi.

According to yet another aspect of the invention, the Gram (−) microorganisms comprise at least one of Enterobacter spp., Escherichia coli, Enterococcus faecalis, Serratia marcescens, Klebsiella pneumoniae, Haemophilus influenzae, Pseudomonas spp., Pseudomonas aeruginosa; Pseudomonas cepacian, Proteus mirabilis, Porphyromonas gingivalis, or Prevotella intermedia.

According to an aspect of the invention, the Gram (+) microorganisms comprise at least one of Acinetobacter spp., Acinetobacter baumannii Staphylococcus spp., Bacillus subtilis, Bacillus cereus, Methicillin Resistant Staphylococcus aureus (MRSA), Staphylococcus aureus, Staphylococcus epidermidis, Streptococcus pyogenes, Streptococcus pneumoniae, or Micrococcus yunnanensis.

According to another aspect of the invention, the fungi comprise at least one of Aspergillus spp., Trichophyton rubrum, Aspergillus spp., A. brasiliensis, A. flavus, A. fumigatus, T. mentagrophytes, T. tonsurans, T. verrucosum, or E. floccosum.

According to yet another aspect of the invention, the yeast comprises at least one of Candida spp., Candida albicans, Candida glabrata, Candida parapsilosis, Candida auris, Candida tropicalis, or Issatchenkia orientalis.

According to still another aspect of the invention, the formulation further comprises an anesthetic agent as an inactive ingredient.

According to an aspect of the invention, the anesthetic agent comprises lidocaine, pramoxine, phenol, prilocaine, benzocaine, dibucaine, bupivacaine, proparacaine, epinephrine, capsaicin, menthol, methyl salicylate, tetracaine, camphor, cocaine, dyclonine, or any combination thereof.

According to another aspect of the invention, the formulation further comprises a disinfectant alcohol as an inactive ingredient.

According to still another aspect of the invention, the disinfectant alcohol comprises Isopropyl alcohol (IPA), ethanol, or n-propanol.

According to yet another aspect of the invention, the formulation further comprises a disinfectant alcohol as an inactive ingredient.

According to another aspect of the invention, disinfectant alcohol comprises Isopropyl alcohol (IPA), ethanol, or n-propanol.

According to an aspect of the invention, skin region of the subject has at least one of an abrasion, a scar, a burn, a scratch, or a cut.

According to another aspect of the invention, skin region is at least one of near a nail of the subject, or on a nail bed of the subject.

According to still another aspect of the invention, the formulation comprises Perillyl alcohol as the active monoterpene, and N-methyl pyrrolidone (NMP), Polyethylene glycol (15)-hydroxystearate, Polyethylene-polypropylene glycol and water as inactive ingredients.

According to yet another aspect of the invention, the formulation comprises Perillyl alcohol as the active monoterpene, and Hydroxypropyl-β-Cyclodextrin (HPbCD), Polyvinylpyrrolidone (PVP) and water as inactive ingredients.

According to an aspect of the invention, the formulation comprises Perillyl alcohol as the active monoterpene, and Isopropyl alcohol, polyacrylic acid, and Isopropyl myristate in water as inactive ingredients.

According to another aspect of the invention, the formulation comprises Perillyl alcohol or perillartine as the active monoterpene, and octadecanol, propanediol, hexadecanol, oleic acid, and mineral oil as inactive ingredients.

According to still another aspect of the invention, the formulation comprises Perillyl alcohol or perillartine as the active monoterpene, and bee's wax, mineral oil, and hexadecanol as inactive ingredients.

According to yet another aspect of the invention, the formulation comprises Perillyl alcohol or perillartine as the active monoterpene, and hydroxypropyl cellulose, polyoxyethylene sorbitan monostearate, DI water, polyacrylic acid., sodium bicarbonate and Isopropyl alcohol as inactive ingredients.

According to a further aspect of the invention, the formulation comprises perillartine as the active monoterpene, and hydroxypropyl methylcellulose, polyacrylic acid, isopropyl myristate 96%, sodium bicarbonate, polyoxyethylene 20-sorbitan monooloeate, isopropyl alcohol, and water as inactive ingredients.

According to still another aspect of the invention, the formulation comprises Perillyl alcohol or perillartine as the active monoterpene, and sodium xylene sulfonate, sodium lauryl sulfate, sodium capryl sulfonate, polyethylene glycol mono (nonylphenyl) ether, isopropyl alcohol and water as inactive ingredients.

According to yet another aspect of the invention, the formulation comprises Perillyl alcohol or perillartine as the active monoterpene, and sodium xylene sulfonate, sodium lauryl sulfate, sodium capryl sulfonate, isopropyl alcohol and water as inactive ingredients.

According to another aspect of the invention, the formulation comprises Perillyl alcohol or perillartine as the active monoterpene, and mineral as inactive ingredient.

According to a further aspect of the invention, the formulation is administered orally to said subject.

According to an aspect of the invention, the formulation comprises Perillyl alcohol or perillartine as the active monoterpene, and sorbitol, Polyoxyethylene sorbitan monostearate, a flavoring agent, and water as inactive ingredients.

According to another aspect of the invention, the formulation comprises Perillyl alcohol or perillartine as the active monoterpene, and Magnesium Stearate, Stearic Acid, and a flavoring agent as inactive ingredients.

According to another aspect of the invention, a formulation comprising at least one monoterpene as an active ingredient for use in reducing pathogenic microorganisms on a subject is provided.

According to still another aspect of the invention, a formulation comprising at least one monoterpene as an active ingredient selected from the group consisting of Perillyl alcohol, Perillaldehyde, Perillyl ester, Perillyl Imine, Perillartine, and combinations thereof, for use in reducing pathogenic microorganisms on a subject is provided.

According to yet another aspect of the invention, a formulation comprising at least one monoterpene as an active ingredient in a concentration between 0.1% to 3%, for use in reducing pathogenic microorganisms on a subject is provided.

According to a further aspect of the invention, a formulation comprising at least one monoterpene as an active ingredient selected from the group consisting of Perillyl alcohol, Perillaldehyde, Perillyl ester, Perillyl Imine, Perillartine, and combinations thereof, in a concentration between 0.1% to 3%, for use in reducing pathogenic microorganisms on a subject is provided.

### BRIEF DESCRIPTION OF DRAWINGS

Further features and advantages of the invention will become apparent from the following detailed description taken in conjunction with the accompanying figures showing illustrative embodiments of the invention, in which:

FIG. 1 illustrates the layers of human or animal epidermis.

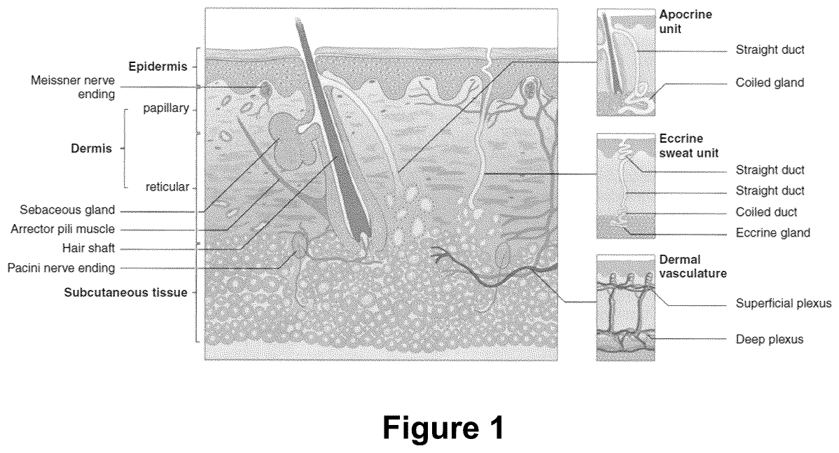

FIG. 2 illustrates a comparison between normal human skin and skin with dermatitis.

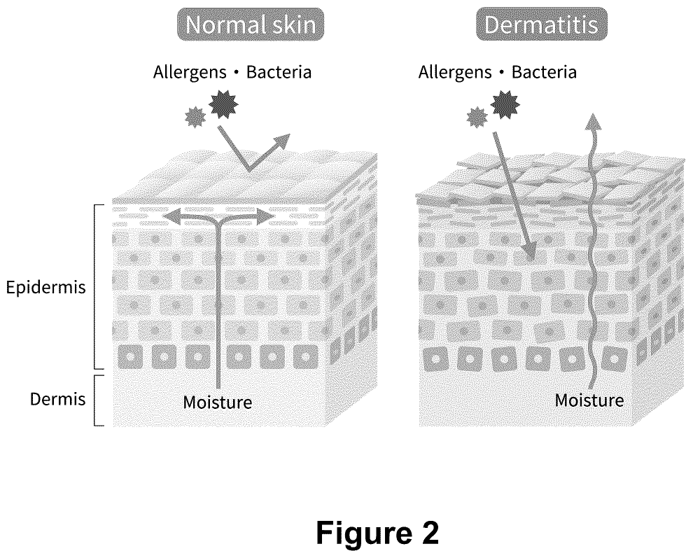

FIG. 3 shows the chemical structure of perillyl alcohol (POH) and other oxidized monoterpenes, according to the present invention.

FIG. 4 shows the metabolic path of perillyl alcohol (POH).

FIG. 5 illustrates the porcine model used to inoculate wounds on a pig with pathogenic microorganisms A. baumannii and Methicillin-Resistant Staphylococcus Aureus (MRSA USA300).

FIG. 6 shows the Staphylococcus Aureus (MRSA USA300) bacterial count on the treated wounds compared to untreated wounds after 24 hours.

FIG. 7 shows the A. baumannii ATCC 19606 bacterial count on the treated wounds compared to untreated wounds after 24 hours.

FIG. 8 shows a comparative assessment between Staphylococcus Aureus (MRSA USA300) and A. baumannii ATCC 19606 bacterial count on the treated wounds compared to untreated wounds after 24 hours.

Throughout the figures, the same reference numbers and characters, unless otherwise stated, are used to denote elements, components, portions or features of the illustrated embodiments. The subject invention will be described in detail in conjunction with the accompanying figures, in view of the illustrative embodiments.

### DETAILED DESCRIPTION OF THE INVENTION

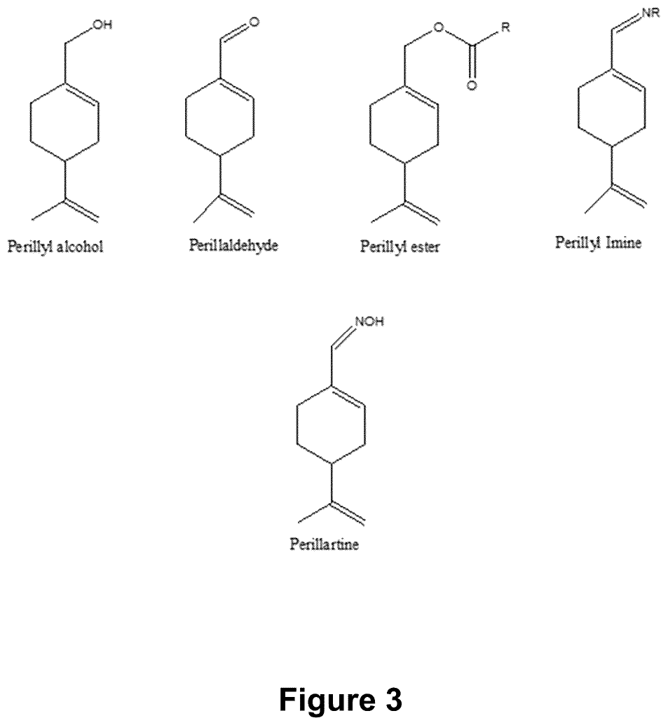

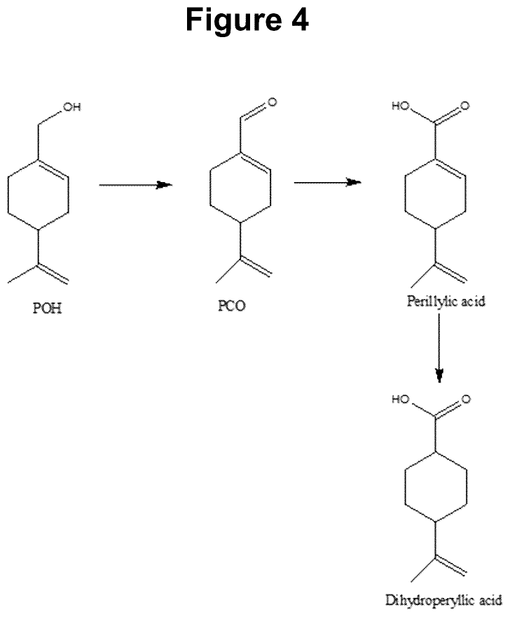

This invention is directed to the capacity of perillyl alcohol (POH) and other oxidized monoterpenes shown in FIG. 3, that remain active when delivered to skin with and without an open wound that has not been cleaned or has been debrided when there is soil, blood, or any other bodily fluid present. Furthermore, it can remain effective against pathogenic microorganisms in the wound for a period of at least 24 hrs. This invention includes perillyl alcohol and derivatives of such (e.g., perillartine, perillyl esters,) as shown below, as well as other prodrugs that someone skilled in the art can design and synthesize to provide a metabolic precursor to perillaldehyde (PCO). Where “R” represents any organic or inorganic molecule that can bond to the indicated atom. The nitrogen molecules include neutral molecules as well as appropriate salts. Specifically, perillyl alcohol has a metabolic path as shown in FIG. 4.

Both POH and PCO exhibit similar antimicrobial properties as demonstrated U.S. Pat. No. 7,157,499 B2 to Zayas et. al., incorporated herein by reference in its entirety. However, once the molecules are oxidized to the acids (Perillic and Dihydroperillic) they lose the antimicrobial activity. On the other hand, the monooxidterpenes according to this invention (FIG. 3) are all precursors to PCO given the fact that all under metabolic conditions will convert the source molecule PCO as in the case of the imine and perillartine type molecule. POH is a precursor to PCO, but it is much more tolerable to the skin and better suited to be formulated to avoid the high vapor pressure of the PCO. This physical characteristic of PCO makes it difficult to maintain a specific concentration in the formulation throughout time due to evaporation. This makes PCO not as commercially viable as POH or derivatives since the latter can stay in the formulation as expected of stable drug products. This approach and use of these “prodrugs” allow the formulation of agents according to application and use as an intact skin and wound treatment.

The antiseptic and antimicrobial actions of oxygenated monoterpenes, such as POH and derivatives, occur at the cell membrane level. As indicated, the oxygenated terpenoids disrupt the membrane via physicochemical changes as they enter the lipid layer. The strongest inhibitory effects exhibited were on microbial oxygen uptake and oxidative phosphorylation. Additional evidence points to reduction of the membrane order, affecting lipid bilayer packing, and increasing membrane small ion permeability or loss of chemiosmotic control. Thus, oxygenated monoterpenes act by adversely affecting three fundamental properties of lipid bilayers, namely stability, order, and permeability.

Since POH and its derivatives according to the invention share the same properties and mechanism of action, a combination of any of these monoterpenes in any ratio, are expected to individually exert their antimicrobial properties with or without enhancing each other's effect, but not minimizing them either.

### In-vitro Tests

As part of the formulation process, the addition of an anesthetic agent is relevant to the treatment of wounds. According to an embodiment of the invention, while formulating the monoterpenes a 0.1% lidocaine was added. The gel turned to a yellow color due to the lidocaine but retained the same antimicrobial activity. An example is provided for the activity against dermatophytes Trichophyton spp. and Epidermophyton floccosum.

TABLE 2
Inhibition Halos per Formulation (Dermatophytes)

| Formulation | T. rubrum | T. mentagrophytes | T. tonsurans | T. verrucosum | E. floccosum |
|---|---|---|---|---|---|
| SDA Control | No growth | | | | |
| Org. Control | No halo | No halo | No halo | No halo | No halo |
| PCO | (0.5 ml) | (0.5 ml) | (0.5 ml) | nm+ | nm+ |
| 1%/Lidocaine | 12.92 mm | 14.41 mm | 14.41 mm | | |
| 0.1% - | (1.0 ml) | (1.0 ml) | (1.0 ml) | nm+ | nm+ |
| (Liquid) | 8.69 mm | 10.76 mm | 14.36 mm | | |

In the above table, nm+ indicates not measurable due to total inhibition.

TABLE 3
Inhibition Halos per Formulation (Bacteria and Fungi)

| Formulation | E. coli | S. aureus | P. aeruginosa | B. subtilis | A. niger | C. albicans |
|---|---|---|---|---|---|---|
| TSA Control | No growth | | | | | |
| Org. Control | No halo | No halo | No halo | No halo | No halo | No halo |
| Perillartine 1% | (1.0 ml) | (1.0 ml) | (1.0 ml) | (1.0 ml) | (1.0ml) | (1.0 ml) |
| (Liquid) | 4.61 mm | 4.65 mm | 3.63 mm | 4.51 mm | 3.53 mm | 2.54 mm |

While lidocaine was used as an anesthetic agent in the above embodiment, it is understood that other topical anesthetic agents are encompassed by the invention to the extent that the formulation retains its antimicrobial activity. Some non-limiting examples of topical anesthetic agents that can be used include pramoxine, phenol, prilocaine, benzocaine, dibucaine, bupivacaine, proparacaine, epinephrine, capsaicin, menthol, methyl salicylate, tetracaine, camphor, cocaine, dyclonine, and combinations thereof. Moreover, according to another embodiment of the invention, the formulation including the anesthetic agent can be also used on skin that has no wounds.

### In-Vivo Studies

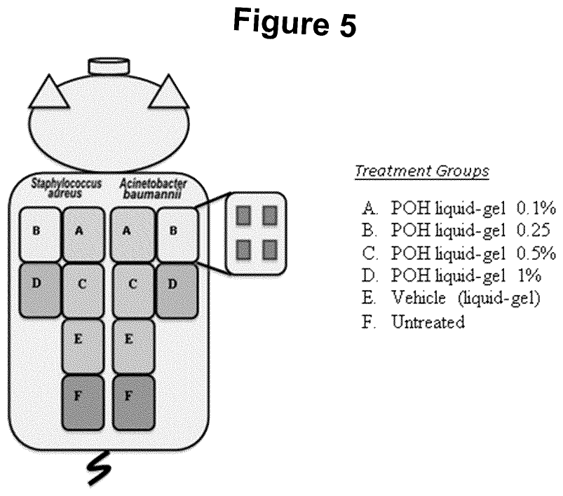

While studying the effects of an alcohol-free formulation containing perillyl alcohol at different concentrations using a porcine model, pathogenic microorganisms A. baumannii and Methicillin-Resistant Staphylococcus aureus (MRSA USA300) were used to inoculate the wounds. One side of the pig was inoculated with MRSA USA300 and the other with A. baumannii (ATCC 19606), as illustrated in FIG. 5.

A. baumannii is a Gram (−) bacteria, which is known as a significant hospital pathogen that has developed significant resistance to antimicrobial agents, and a leading cause of hospital acquired infections, specifically bloodstream infections (BSI) (Jasna Hrenovic et. al., Applied and Environmental Microbiology p. 2860-2866 May 2014 Volume 80 Number 9). It has also been shown to be multi-drug resistant to carbapenem and ampicillin-sublactam antibiotics (CASR) (Teena Chopra et. al., Antimicrobial Agents and Chemotherapy p. 6270-6275 Dec. 2013 Volume 57 Number 12). MRSA USA 300 is methicillin-resistant S. aureus, a Gram (+) microorganism that is a common cause of hospital-acquired pneumonia (HAP) and ventilator-associated pneumonia (VAP) (Jakub M Kwiecinski et. al., Curr Opin Microbiol. 2020 February; 53:51-60. doi: 10.1016/j.mib.2020.02.005). This opportunistic pathogen is the main cause of sepsis and endocarditis due to blood-stream infections.

As a result of the in-vivo study above, the expectation was that the Perillyl Alcohol would be deactivated, as would be evidenced by growth of the microorganisms in the exposed skin, and the bacterial count would grow, or at the very least remain the same to show a bacteriostatic effect. This assumption was based on the mechanism of action proposed for Perillyl Alcohol as discussed below.

The antiseptic actions of oxygenated terpenoids, such as Perillyl Alcohol, are proposed at the cell membrane level. Considerable research demonstrates that oxygenated terpenoids disrupt the membrane via physicochemical changes as they enter the lipid layer. The strongest inhibitory effects exhibited were on microbial oxygen uptake and oxidative phosphorylation. Additional evidence points to reduction of the membrane order, affecting lipid bilayer packing, and increasing membrane small ion permeability or loss of chemiosmotic control. Thus, oxygenated terpenoids act by adversely affecting three fundamental properties of lipid bilayers, namely stability, order, and permeability.

However, to our surprise, the complete opposite effect and unexpected result was observed. Based on the proposed mechanism of action of perillyl alcohol and derivatives, the red blood cells would have been expected to be attacked, and through this process inactivate the disinfectant. The reason is that the red blood cell membrane is essentially a two-dimensional (2D) structure, comprised of a cytoskeleton and a lipid bilayer, tethered together. The lipid bilayer includes various types of phospholipids, sphingolipids, cholesterol, and integral membrane proteins, such as band-3 and glycophorin. So based on the proposed mechanism of action of the monoterpene, the bilayer of the red blood cells should have been affected by the perillyl alcohol and should have resulted in inactivation of the disinfectant effect.

Nevertheless, perillyl alcohol showed to be selective towards cells from microorganisms and differentiate over other cells or proteins present that could deactivate its disinfectant properties. Furthermore, blood contains several enzymes that offer potential metabolization of perillyl alcohol. Prior systemic studies using perillyl alcohol have failed to deliver the compound via its metabolization before it can reach the therapeutic effect. It has been shown that perillyl alcohol metabolizes quickly in rats where it was not detected. In separate studies, it was shown that in humans, dogs, and rats, it rapidly metabolized showing that it could have been affected by enzymes.

### Treatment Regimen (Porcine Model)

Forty-eight (48) rectangular wounds measuring 10 mm×7 mm×0.5 mm deep were made in the paravertebral and thoracic area with a specialized electrokeratome fitted with a 7 mm blade. Wounds were blotted dry with sterile gauze pads. The wounds were separated from one another by 15 mm of unwounded skin and were individually dressed. As illustrated in FIG. 5, four (4) wounds were randomly assigned to each of the six (6) treatment groups (A-F), and they were then inoculated according to the experimental design shown in FIG. 5. One side of the animal was inoculated with Methicillin Resistant Staphylococcus aureus (USA 300) and the other side with Acinetobacter baumannii (ATCC 19606).

Immediately after performing the incisions, the wounds were inoculated with the appropriate bacterial strain as described in the “Wound Inoculation” section below and treated within 30 minutes of inoculation. Wounds were divided into six (6) groups per bacteria of four wounds each, treated with the appropriate treatments provided by sponsor (see design above FIG. 5). All wounds were covered, individually, with a polyurethane film dressing (Tegaderm; 3M, St. Paul, MN) which were secured along the edges using surgical tape. All dressings were covered and secured by wrapping the animal with self-adherent elastic bandages (Coban; 3M, St. Paul, MN). The dressings were left in place for 24 hours to determine the treatment efficacy in decreasing bacterial proliferation in the wounds. After 24 hours, the dressings were removed, and four wounds were recovered for each treatment group as described below.

### Microbiological Techniques

### Wound Inoculation

A fresh culture of Methicillin Resistant Staphylococcus aureus (NRS 384/USA 300) and Acinetobacter baumannii (ATCC 19606) were used in this study. The challenge inoculum suspension was prepared by scraping the overnight growth from a culture plate into 4.5 mL of sterile water. This resulted in a suspension concentration of approximately 108 colony forming units/mL (CFU/mL) for each bacterium. Serial dilutions were made until a concentration of 106 colony forming units/mL (CFU/mL) was achieved as determined by optical density measurements. The inoculum was vortexed, and 25 mL of the suspension was inoculated into each wound. In addition, serial dilutions of the suspension were plated onto culture media to quantify the exact concentration of viable organisms used for this experiment.

### Recovery Methods

### Wound Assessment

Four wounds from each treatment group (A-F) were recovered 24 hours after treatment. A sterile surgical steel cylinder (22 mm inside diameter) was placed around the wound area. One (1) mL of all-purpose neutralizer solution was pipetted into the cylinder and the site was scrubbed with a sterile Teflon spatula for 30 seconds. Serial dilutions were made from all culture samples and the extent of microbiological contamination assessed using the Spiral Plater System (Spiral Biotech, Norwood, MA). This system deposits a 50 μl aliquot of the scrub bacterial suspension over the surface of a rotating agar plate. ORSAB Screening Agar Base with Selective Supplement was used to isolate Methicillin Resistant Staphylococcus aureus USA 300 from the wounds, and Leeds Acinetobacter medium was used to isolate Acinetobacter baumannii (ATCC 19606). All plates were incubated aerobically overnight (16-24 hours) at 37° C., after which the number of viable colonies were counted.

### Observations

Observations were taken during the treatment application and during the assessment time at 48 hours. Within 30 minutes of inoculation, 500 μL of the appropriate treatments were deposited into each wound respectively and spread with sterile spatula.

### Results

After counting the colonies, the data was tabulated and the Log of colony forming units/mL (Log CFU/mL) for Methicillin Resistant Staphylococcus aureus (MRSA) USA300 and Acinetobacter baumannii ATCC 19606 (AB19606) determined. The arithmetic mean of the Log (CFU/mL) and standard deviation were calculated for each treatment. The effect of the treatments on the challenge pathogen was determined. Table 4 and Table 5 present the raw data.

Inoculation with Staphylococcus aureus (MRSA) USA300

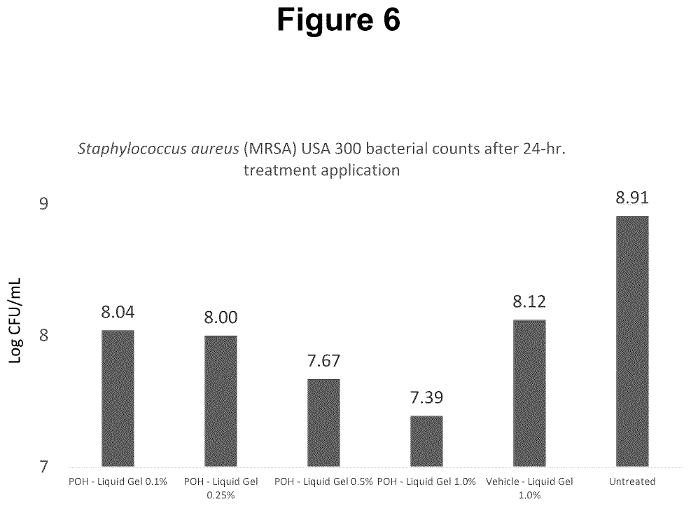

When examining each treatment group, it was determined that the wounds treated with Perillyl Alcohol liquid gel 1% (treatment D) showed the lowest Log CFU/mL (7.39±0.35) MRSA USA 300 compared to other treatment groups (see FIG. 6). This was followed by treatment (C) Perillyl Alcohol liquid gel 0.5% (7.67±0.36), treatment (B) Perillyl Alcohol liquid gel 0.25% (8.00±0.06), treatment (A) Perillyl Alcohol liquid gel 0.1% (8.04±0.17), and treatment (E) Vehicle liquid gel (8.12±0.15) of MRSA USA 300 recovered from wounds. Untreated group wounds resulted in a greater Log CFU/mL (8.91±0.35) of MRSA USA300.

After 24 hours all treatment groups assessment reduced bacterial count compared to untreated group (treatment F), as shown in FIG. 6. Wounds treated with treatment D (Perillyl Alcohol liquid gel 1%) showed the largest reduction of MRSA (1.52±0.18 Log CFU/mL reduction) compared to treatment F (Untreated group). These values corresponded to a 96.98% reduction of MRSA at 24 hours when compared to wounds treated with treatment F (Untreated group). Bacterial reduction in wounds treated with Treatment B (Perillyl Alcohol liquid gel 0.25%) and treatment A (Perillyl Alcohol liquid gel 0.1%) had similar percentage reduction (87.70% and 86.51%, respectively) of MRSA after 24 hours assessment compared to untreated wounds (treatment F).

Inoculation with Acinetobacter baumannii (ATCC 19606)

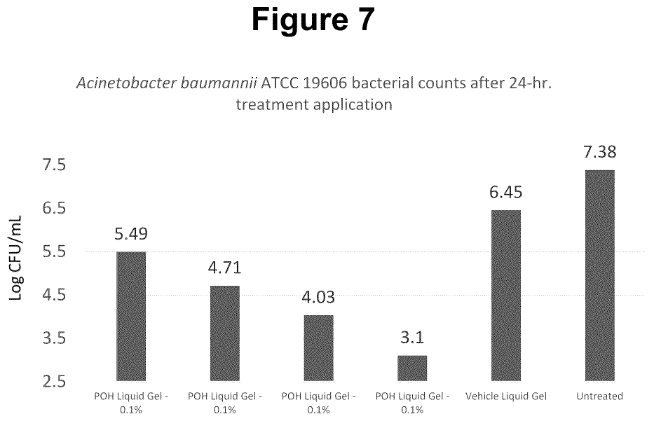

Wounds treated with treatment D (Perillyl Alcohol liquid gel 1%) showed the lowest Log CFU/mL (3.10±0.56) of AB19606 when compared to other treatment groups (see FIG. 7). This was followed by treatment (C) Perillyl Alcohol liquid gel 0.5% (4.03±0.01), treatment (B) Perillyl Alcohol liquid gel 0.25% (4.71±0.32), treatment (A) Perillyl Alcohol liquid gel 0.1% (5.49±0.30), and treatment (E) Vehicle liquid gel (6.45±0.43) AB19606 recovered from wounds. Untreated group wounds resulted in a greater Log CFU/mL (7.38±0.28) of AB19606.

After 24 hours all treatment groups assessment shows reduced bacterial count when compared to untreated wounds (treatment F), as shown in FIG. 7. Wounds treated with treatment D (Perillyl Alcohol liquid gel 1%) showed the largest reduction of AB19606 (4.28±0.28 and 3.35±0.13 Log CFU/mL reduction) compared to untreated wounds (treatment F) and Vehicle liquid-gel (treatment E), respectively. These values corresponded to a 99.99% and 99.96% reduction of AB19606 after 24 hours treatment application when compared to untreated wounds (treatment F) or treated with the vehicle liquid-gel (treatment E), respectively.

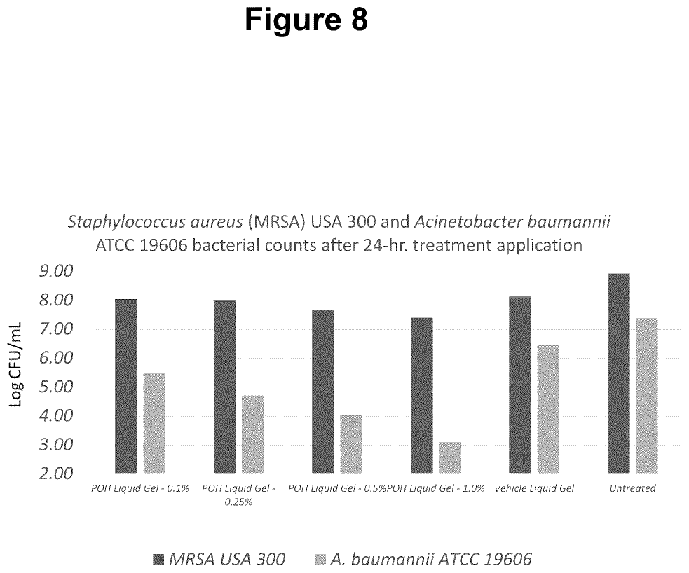

Wounds treated with treatment D (Perillyl Alcohol liquid gel 1%) showed the lowest Log CFU/mL of Methicillin Resistant Staphylococcus aureus (MRSA) USA300 and Acinetobacter baumannii ATCC 19606 when compared to other treatment groups (see FIG. 8). The same trend was observed when the concentrations of Perillyl Alcohol liquid gel increased.

The following trends were noted. Wounds treated with Treatment D (Perillyl Alcohol liquid gel 1%) had the lowest amount of Methicillin Resistant Staphylococcus aureus (MRSA) USA300 and Acinetobacter baumannii (ATCC 19606) recovered 24 hours after treatment application. Wounds treated with Treatment D (Perillyl Alcohol liquid gel 1%) and Treatment C (Perillyl Alcohol liquid gel 0.5%) showed a larger reduction Methicillin Resistant Staphylococcus aureus (MRSA) USA300 and Acinetobacter baumannii (ATCC 19606) as compared to untreated wounds (1.52±0.18 and 4.28±0.28 Log CFU/mL reduction, respectively).

After 24 hours, assessment wounds which were treated with Treatment D (Perillyl Alcohol liquid gel 1%) showed 96.98% and 99.99% reduction of Log CFU/mL of Methicillin Resistant Staphylococcus aureus (MRSA) USA300 and Acinetobacter baumannii (ATCC 19606), respectively when compared to untreated wounds (treatment F). All Perillyl Alcohol liquid gel at different concentrations were more effective against Acinetobacter baumannii than MRSA (FIG. 8).

Overall, the Perillyl Alcohol liquid gel is the most effective in reducing bacterial counts against Acinetobacter baumannii Gram (−) bacteria than Staphylococcus aureus (MRSA) USA300 Gram (+) bacteria. These formulations would also be effective against Pseudomonas aeruginosa Gram (−) bacteria (another common pathogen with higher incidence in infections).

TABLE 4
MRSA USA 300 S. aureus Data

| Bacteria | Dilution | Count | CFU/mL | Log CFU/mL |
|---|---|---|---|---|
| S. aureus | −4 | 44 | 8.79E+06 | 6.94 |
| MRSA USA300 | | | | |

| | | Dilu- | | | Log | |
|---|---|---|---|---|---|---|
| Treatment | Wound | tion | Count | CFU/mL | CFU/mL | |
| A - Perillyl | 1 | −5 | 48 | 9.59E+07 | 7.98 | |
| Alcohol | 2 | −5 | 60 | 1.20E+08 | 8.08 | |
| liquid- | 3 | −5 | 86 | 1.72E+08 | 8.24 | |
| gel 0.1% | 4 | −5 | 35 | 7.00E+07 | 7.84 | STDV |
| | | | Mean | 1.14E+08 | 8.04 | 0.17 |
| B - Perillyl | 1 | −5 | 49 | 9.79E+07 | 7.99 | |
| Alcohol | 2 | −5 | 58 | 1.16E+08 | 8.06 | |
| liquid- | 3 | −5 | 52 | 1.04E+08 | 8.02 | |
| gel 0.25% | 4 | −5 | 41 | 8.20E+07 | 7.91 | STDV |
| | | | Mean | 1.00E+08 | 8.00 | 0.06 |
| C - Perillyl | 1 | −4 | 114 | 2.28E+07 | 7.91 | |
| Alcohol | 2 | −5 | 23 | 4.60E+07 | 7.66 | |
| liquid- | 3 | −5 | 44 | 8.79E+07 | 7.94 | |
| gel 0.5% | 4 | −4 | 73 | 1.46E+07 | 7.16 | STDV |
| | | | Mean | 4.28E+07 | 7.67 | 0.36 |
| D - Perillyl | 1 | −5 | 23 | 4.60E+07 | 7.66 | |
| Alcohol | 2 | −4 | 38 | 7.60E+06 | 6.88 | |
| liquid- | 3 | −5 | 20 | 4.00E+07 | 7.60 | |
| gel 1.0% | 4 | −4 | 131 | 2.62E+07 | 7.42 | STDV |
| | | | Mean | 3.00E+07 | 7.39 | 0.35 |
| E - Vehicle | 1 | −5 | 81 | 1.62E+08 | 8.21 | |
| (liquid- | 2 | −5 | 40 | 8.00E+07 | 7.90 | |
| gel) | 3 | −5 | 74 | 1.48E+08 | 8.17 | |
| | 4 | −5 | 82 | 1.64E+08 | 8.21 | STDV |
| | | | Mean | 1.39E+08 | 8.12 | 0.15 |
| F - Untreated | 1 | −6 | 47 | 9.39E+08 | 8.97 | |
| | 2 | −6 | 62 | 1.24E+09 | 9.09 | |
| | 3 | −6 | 39 | 7.80E+08 | 8.89 | |
| | 4 | −6 | 24 | 4.80E+08 | 8.68 | STDV |
| | | | Mean | 8.60E+08 | 8.91 | 0.17 |

TABLE 5
A. baumannii Data

| Bacteria | Dilution | Count | CFU/mL | Log CFU/mL |
|---|---|---|---|---|
| A. baumannii | −4 | 41 | 8.20E+06 | 6.91 |
| ATCC 19606 | | | | |

| | | Dilu- | | | Log | |
|---|---|---|---|---|---|---|
| Treatment | Wound | tion | Count | CFU/mL | CFU/mL | |
| A - Perillyl | 1 | −2 | 185 | 3.70E+05 | 5.57 | |
| Alcohol | 2 | −3 | 27 | 5.40E+05 | 5.73 | |
| liquid- | 3 | −3 | 21 | 4.20E+05 | 5.62 | |
| gel 0.1% | 4 | −2 | 56 | 1.12E+05 | 5.05 | STDV |
| | | | Mean | 3.61E+05 | 5.49 | 0.30 |
| B - Perillyl | 1 | −2 | 54 | 1.08E+05 | 5.03 | |
| Alcohol | 2 | −2 | 31 | 6.20E+04 | 4.79 | |
| liquid- | 3 | −2 | 28 | 5.60E+04 | 4.75 | |
| gel 0.25% | 4 | −1 | 94 | 1.88E+04 | 4.27 | STDV |
| | | | Mean | 6.12E+04 | 4.71 | 0.32 |
| C - Perillyl | 1 | −1 | 52 | 1.04E+04 | 4.02 | |
| Alcohol | 2 | −1 | 54 | 1.08E+04 | 4.03 | |
| liquid- | 3 | −1 | 52 | 1.04E+04 | 4.02 | |
| gel 0.5% | 4 | −1 | 55 | 1.10E+04 | 4.04 | STDV |
| | | | Mean | 1.07E+04 | 4.03 | 0.01 |
| D - Perillyl | 1 | −1 | 39 | 7.80E+03 | 3.89 | |
| Alcohol | 2 | 0 | 60 | 1.20E+03 | 3.08 | |
| liquid- | 3 | 0 | 32 | 6.40E+02 | 2.81 | |
| gel 1.0% | 4 | 0 | 21 | 4.20E+02 | 2.62 | STDV |
| | | | Mean | 2.52E+03 | 3.10 | 0.56 |
| E - Vehicle | 1 | −3 | 79 | 1.58E+06 | 6.20 | |
| (liquid- | 2 | −4 | 26 | 5.20E+06 | 6.72 | |
| gel) | 3 | −4 | 40 | 8.00E+06 | 6.90 | |
| | 4 | −3 | 49 | 9.79E+05 | 5.99 | STDV |
| | | | Mean | 3.94E+06 | 6.45 | 0.43 |
| F - Untreated | 1 | −4 | 87 | 1.74E+07 | 7.24 | |
| | 2 | −4 | 92 | 1.84E+07 | 7.26 | |
| | 3 | −4 | 81 | 1.62E+07 | 7.21 | |
| | 4 | −5 | 31 | 6.20E+07 | 7.79 | STDV |
| | | | Mean | 2.85E+07 | 7.38 | 0.28 |

Without alcohol, the Perillyl Alcohol liquid-gel reduced both microorganisms in one treatment. The 1% formulation containing surfactants and alcohol-free showed a 96.98% and 99.99% reduction of Log CFU/mL of MRSA and A. baumannii after a single 24-hour treatment, respectively, compared to untreated wounds (FIG. 8). Thus, according to the present invention, Perillyl Alcohol remains active in the presence of bodily fluids, blood, and soil (organic or inorganic), with no alcohol. Of particular importance is that this level of reduction in microorganisms is possible in only one treatment.

Furthermore, this also represents a unique behavior for treatment of wounds. This reduction in Gram (+) and Gram (−) microorganisms translates into a shorter treatment for infections on wounds. Since the reduction is quite significant after a 24-hr period, it means that under clinical conditions less applications of Perillyl Alcohol completely render the wound virtually free from infection leading to better outcomes. It also provides relief from the extended use of saline solutions in trauma centers, which is the only current treatment to the disinfection of open wounds.

Most relevant is the fact that the main disinfectant for wounds is the prescription of oral (systemic) antibiotics which only act against bacteria and will generally take days to be effective. In marked contrast, Perillyl Alcohol, as well as other oxidized monoterpenes, have been shown to be active against pathogenic yeast and fungi, as well as dermatophytes and can act immediately preventing potential amputations.

The formulation remains active against yeasts, such as Candida spp. (e.g., C. albicans, C. glabrata, C. parapsilosis). It is known that a main cause of bloodstream infections is Candida spp., which produces Candidemia. Candida infection can spread from your bloodstream to other parts of your body (such as your eyes, kidney, liver, and brain). This phenomenon is known as Invasive Candidemia.

Microorganisms treated with the formulations are listed on Tables 6-9 below.

TABLE 6
Gram Negative

Enterobacter spp.
Escherichia coli
Enterococcus faecalis
Serratia marcescens
Klebsiella pneumoniae
Haemophilus influenzae
Pseudomonas spp.
Pseudomonas aeruginosa
Pseudomonas cepacia
Proteus mirabilis
Porphyromonas gingivalis
Prevotella intermedia

TABLE 7
Gram positive

Acinetobacter spp.
Acinetobacter baumannii
Staphylococcus spp.
Bacillus subtilis
Bacillus cereus
Methicillin Resistant
Staphylococcus aureus
(MRSA)
Staphylococcus aureus
Staphylococcus epidermidis
Streptococcus pyogenes
Streptococcus pneumoniae
Micrococcus yunnanensis

TABLE 8
Fungi

Aspergillus spp.
Trichophyton rubrum
Aspergillus spp.
A. brasiliensis
A. flavus
A. fumigatus
T. mentagrophytes
T. tonsurans
T. verrucosum
E. floccosum

TABLE 9
Yeast

Candida spp.
Candida albicans
Candida glabrata
Candida parapsilosis
Candida auris
Candida tropicalis
Issatchenkia orientalis

Relating to the fungal species targeted by our research are those most recovered from humans; these include:
1. T. mentagrophytes, T. rubrum and T. verrucosum as causative agents of Tinea barbae
2. T. tonsurans as causative agent of Tinea capitis
3. T. mentagrophytes, T. rubrum and E. floccusum as causative agents of Tinea pedis
4. T. mentagrophytes and T. rubrum as causative agents of Tinea corporis
5. T. mentagrophytes, T. rubrum and E. floccusum as causative agents of Tinea cruris
6. T. mentagrophytes and T. rubrum as causative agents of Tinea manuum
7. T. mentagrophytes, T. rubrum and E. floccusum as causative agents of Tinea unguium

For example, a solution of perillartine (1,4 cyclohexadiene-1-carboxaldehyde syn-oxime) was used to treat different dermatophytes with the results shown in Figure V below combining different volumes to mimic dilution:

TABLE 10
Inhibition Halos per Formulation (Dermatophytes)

| Formulation | T. rubrum | T. mentagrophytes | T. tonsurans | T. verrucosum | E. floccosum |
|---|---|---|---|---|---|
| SDA Control | No growth | | | | |
| Org. Control | No halo | No halo | No halo | No halo | No halo |
| Perillartine 1% | (0.5 ml) | (0.5 ml) | (0.5 ml) | (0.5 ml) | (0.5 ml) |
| (Liquid) | 14.78 mm | 20.84 mm | nm+ | nm+ | 17.50 mm |
| | (1.0 ml) | (1.0 ml) | (1.0 ml) | (1.0 ml) | (1.0 ml) |
| | 16.70 mm | 17.37 mm | nm+ | nm+ | 15.34 mm |

In the above table, nm+ indicates not measurable due to total inhibition.

When treating microorganisms in samples of saliva from healthy volunteers, we found that although perillartine is a semi-synthetic sweetener it also acts as an antibacterial in the mouth, as shown in Table 11.

TABLE 11
Inhibition Halos per Formulation (Bacteria in Mouth)

| Formulation | Volunteer #1 | Volunteer #2 | Volunteer #3 |
|---|---|---|---|
| TSA Control | No growth | | |
| Perillartine 1% | 4.33 mm | 5.19 mm | 4.61 mm |
| (Liquid) | | | |
| Chlorhexidine | 5.33 mm | 5.70 mm | 5.43 mm |
| gluconate | | | |
| 0.12% | | | |
| (Liquid) | | | |
| Listerine | 0.91 mm | 1.10 mm | 0.83 mm |
| (Liquid) | | | |

Finally, the third group of microorganisms included in our research was tested by using the agar diffusion-disk technique. Four different bacteria and two different fungi were pour plated individually and the anti-microbial activity was detected either by inhibiting all growth or by showing a distinguished halo around a diffusion-disk previously impregnated with the formulation. Halo formation was measured with a caliper and biological activity was considered according to size. The formulation containing perillartine was exposed to aliquots of 1.0 ml of each organism, from an initial inoculum's concentration greater than 10⁴ cfu/ml. Details of specific applications and formulations are set forth below in Table VII, which summarizes the various results obtained.

TABLE 12
Inhibition Halos per Formulation (Bacteria and Fungi)

| Formulation | E. coli | S. aureus | P. aeruginosa | B. subtilis | A. brasiliensis | C. albicans |
|---|---|---|---|---|---|---|
| TSA Control | No growth | | | | | |
| Org. Control | No halo | No halo | No halo | No halo | No halo | No halo |
| Perillartine 1% | (1.0 ml) | (1.0 ml) | (1.0 ml) | (1.0 ml) | (1.0 ml) | (1.0 ml) |
| (Liquid) | 4.61 mm | 4.65 mm | 3.63 mm | 4.51 mm | 3.53 mm | 2.54 mm |

### Formulations Tested

The formulations tested contain solvents that are not considered polymers, surfactants, such as N-methyl pyrrolidone (NMP) is used as a solubilizing agent. These series of preferred formulations are liquid under refrigeration (approximately 4° C.) and when warmed above 25° C. become a semisolid solution or liquid-gel

### (Table 13).

TABLE 13
Preferred Formulation Alcohol-free Solution of (S)-Perillyl Alcohol

| | |
|---|---|
| Solvent: | 5% NMP:5% Solutol HS15:14% Pluronic F-127:76% |
| | Water |
| Concentration: | 0.1 to 1% |
| Physical State: | Solution |

Another version of the preferred formulations contains Hydroxypropyl-β-Cyclodextrin (HPbCD). HPbCD is considered an enabling excipient, not a solvent, preservative, emollient, sunscreen, emulsifier, gelling agent, thickening agent, humectant, sequestrant, or polymer. (Table 14)

TABLE 14
Preferred Formulation Alcohol-free Solution of (S)-Perillyl Alcohol

| | |
|---|---|
| Solvent: | 20% HPbCD:3% Povidone K90:77% Water |
| Concentration: | 0.1 to 1% |
| Physical State: | Solution |

The above solution listed on Table 14, also has the property of remaining liquid while stored under refrigeration, and when warmed up to room temperature will become a liquid-gel.

When comparing CHG to the effectiveness of POH, other preferred formulations provided proof of comparable effectiveness when evaluating minimum-inhibitory concentration and minimum bactericidal concentration (MIC/MBC) results (Tables 16-18). The inclusion of IPA is mandatory by the Food and Drug Administration's (FDA) requirements as part of the comparison with the chlorhexidine solution. As shown in the study for open wounds in a porcine model (above), IPA is not necessary to achieve the antimicrobial effect. The IPA was formulated to maintain it below flammability levels. CHG on the other hand must be formulated using 70% IPA to provide antimicrobial effectiveness. One of ordinary skill in the art would appreciate that other disinfectant alcohols such as but not limited to ethanol, or n-propanol can also be used alone or in combination with IPA in accordance with the teachings of the present invention.

The antimicrobial effectiveness of POH diminishes in more dilute solutions. Therefore, challenges to its effectiveness were performed at 0.1% concentration and found to be reasonable against all microorganisms. At higher concentrations, over 90% concentrations it was observed to present immediate effects eradicating the microorganisms completely. However, POH at concentrations equal to and higher than 4% will cause skin irritation and thus are not practical for clinical applications. Therefore, the highest concentration proposed is 3% to maintain a practical clinical range.

TABLE 15
Preferred Formulation 0.1 to 3% Solution
of (S)-Perillyl Alcohol and 15% IPA

| | |
|---|---|
| Solvent: | 1% Yellow 6:0.8% Carbopol 940 (Polyacrylic acid) |
| | (Lubrizol):15% Isopropanol:0.08% Isopropyl |
| | myristate in Water, final pH 6 adjusted with NaOH |
| Physical State: | Opaque orange thixotropic gel |

TABLE 16
MIC Alcohol Containing Formulation
(1% Perillyl Alcohol/15% IPA)

| Minimum Inhibitory | | 1% POH/ | |
|---|---|---|---|
| Concentration | CHG | 15% IPA | Vehicle |
| Candida albicans | 1:1024 | 1:16 | <1:2 |
| ATCC #10231 | | | |
| Enterococcus faecalis | 1:8192 | 1:16 | <1:2 |
| ATCC #29212 | | | |
| Escherichia coli | 1:8192 | 1:16 | <1:2 |
| ATCC #25922 | | | |
| Klebsiella pneumoniae | 1:4096 | 1:16 | <1:2 |
| ATCC #10031 | | | |
| Micrococcus yunnanensis | 1:8192 | 1:16 | <1:2 |
| (formerly luteus) | | | |
| ATCC #7468 | | | |
| Proteus mirabilis | 1:512 | 1:16 | <1:2 |
| ATCC #7002 | | | |
| Pseudomonas aeruginosa | 1:2048 | 1:16 | <1:2 |
| ATCC #27853 | | | |
| Serratia marcescens | 1:512 | 1:16 | <1:2 |
| ATCC #14756 | | | |
| Staphylococcus aureus | 1:8192 | 1:16 | <1:2 |
| MRSA ATCC #33591 | | | |
| Staphylococcus epidermidis | 1:8192 | 1:16 | <1:2 |
| ATCC #12228 | | | |

TABLE 17
MBC Formulation (1% Perillyl Alcohol/15% IPA)

| Minimum Bactericidal | | 1% POH/ | |
|---|---|---|---|
| Concentration | CHG | 15% IPA | Vehicle |
| Candida albicans | 1:1024 | 1:16 | <1:2 |
| ATCC #10231 | | | |
| Enterococcus faecalis | <1:2048 | 1:8 | <1:2 |
| ATCC #29212 | | | |
| Escherichia coli | 1:64 | 1:8 | <1:2 |
| ATCC #25922 | | | |
| Klebsiella pneumoniae | 1:4096 | 1:16 | <1:2 |
| ATCC #10031 | | | |
| Micrococcus yunnanensis | <1:2048 | 1:8 | <1:2 |
| (formerly luteus) | | | |
| ATCC #7468 | | | |
| Proteus mirabilis | 1:512 | 1:8 | <1:2 |
| ATCC #7002 | | | |
| Pseudomonas aeruginosa | 1:1024 | 1:8 | <1:2 |
| ATCC #27853 | | | |
| Serratia marcescens | 1:256 | 1:8 | <1:2 |
| ATCC #14756 | | | |
| Staphylococcus aureus | 1:4096 | 1:8 | <1:2 |
| MRSA ATCC #33591 | | | |
| Staphylococcus epidermidis | 1:8192 | 1:8 | <1:2 |
| ATCC #12228 | | | |

TABLE 18
Time-Kill (1% Perillyl Alcohol/15% IPA).

| Microorganism | ChloraPrep ® | 1% POH/15% IPA |
|---|---|---|
| (Exposure: 30 sec, | (Lot Number: | (Lot Number: |
| 1, 3, 5, 10 minutes) | 83028) | ASLP 05072015) |
| Candida albicans ATCC #10231 | 99.9999% | 99.9999% |
| Enterococcus faecalis | | |
| ATCC #29212 | | |
| Micrococcus yunnanensis | | |
| ATCC #7468 | | |
| Staphylococcus aureus MRSA | | |
| ATCC #33591 | | |
| Staphylococcus epidermidis | | |
| ATCC #12228 | | |
| Proteus mirabilis ATCC #7002 | 99.9999% | 99.9997%* |
| Pseudomonas aeruginosa | 99.9992% | 99.9992% |
| (ATCC 27853) | | |
| Escherichia coli ATCC #25922 | 99.9999% | 99.9992%* |
| Klebsiella pneumoniae | | |
| ATCC #10031 | | |
| Serratia marcescens | | |
| ATCC #14756 | | |

*Due to neutralization being achieved, in part by dilution, the lowest reliable dilution in the Time Kill Assay is 10⁻².

It is important to point out that CHG 2% is a solution that contains 70% isopropyl alcohol (IPA) which is a superb antiseptic in compositions containing greater than 65% concentration. Some studies have concluded that the overwhelming antiseptic effect from CHG is derived from the isopropyl alcohol. A comparative study between a formulation containing 70% isopropyl alcohol versus a 2% CHG commercial solution showed equivalent effectiveness between both products affording an approval by the Food and Drug Administration (Table 19).

TABLE 19
Results from 70% IPA in vehicle versus 2.0%
ChloraPrep ® (in 70% IPA).

| | |
|---|---|
| Body | Mean Log₁₀ CFU/cm² Reduction from Baseline |

| Area | Treatment | 30 sec | 10 min | 6 hours |
|---|---|---|---|---|
| Abdomen | IPA in vehicle | 2.66 | 3.38 | 2.42 |
| | ChloraPrep ® | 2.91 | 3.35 | 2.23 |
| | Vehicle | 1.03 | 1.39 | 1.26 |
| | Normal saline | 0.64 | 0.78 | 0.94 |
| Groin | IPA in vehicle | 3.56 | 4.28 | 3.01 |
| | ChloraPrep ® | 3.36 | 4.2 | 2.72 |
| | Vehicle | 1.24 | 1.83 | 1.98 |
| | Normal saline | 1.2 | 1.43 | 1.6 |

*Randomized, paired comparison study - 320 volunteers.

The formulations according to the present invention can be used for application of the antimicrobials to wounds, and in addition to semisolid delivery systems (e.g., gels, cream, ointment, foam), formulated as immediate release the invention also includes extended and/or sustained release systems using ingredients that someone skilled in the art can formulate.

These formulations include but are not limited to microencapsulation processes, application of the use of nanoparticles, or transdermal patches. These systems can include an anesthetic (e.g., lidocaine) as part of the formulation meant to provide temporary relief of pain. The same technology can be applied if formulated as a foam to provide occlusion and physical protection of the wound while providing disinfection. In addition, the delivery system can include liquid systems for irrigation such as flushing a wound to remove dead and necrotic tissue while at the same time providing immediate disinfection of the affected area. It can also be used during surgical procedures to maintain a clean wound and prevent surgical site infections.

Typical formulations can include excipients for a combination of the POH or derivative alone, or with anesthetic (e.g., lidocaine), or POH or derivative plus alcohol (e.g., IPA, ethanol), or POH or derivative plus alcohol plus anesthetic in gel, cream, ointment, foam, time-release, microencapsulation, nanoparticles. These formulations are meant to deliver the antimicrobial prophylactic and therapeutic effect of the POH or derivatives alone or in combination with an alcohol (e.g., IPA, ethanol), with anesthetic, or with anesthetic and alcohol to the affected area including disinfection of the intact skin, as well as open wounds, in the presence of organics (e.g., bodily fluids), inorganic (e.g., dirt, soil), blood, or soap. For the purpose of the invention, the formulation is administered to the skin region of a subject needing disinfection, for either prophylactic reasons (e.g., disinfecting an intact skin region prior to a surgical procedure, disinfecting a nail area or as a hand disinfectant) or therapeutic reasons (e.g., disinfecting an open wound or a cut). Thus, for the purpose of the invention, the term “in need thereof” includes both situations, the need to prevent possible infection on a skin region and the need to reduce or eliminate infection on a skin region already infected. Moreover, one of ordinary skill in the art would understand that an effective amount of the formulation is determined at least on the size of the skin region, the depth of a wound or cut on the skin and level of infection on the skin region. among other considerations.

For formulation in gels can include gelling agents, stabilizers, dispersing agents, penetration enhancers, buffers, and preservatives, as well as excipients that provide solvent action and humectant properties. They can also be prepared in cream form by addition of thickeners, emulsifying agents, preservatives, antioxidants, and buffer agents, and those ingredients as included in the current version of the United States Pharmacopeia, Europe Pharmacopoeia, Japan and other international standards such as the International Pharmaceutical Excipients Council (IPEC) selected and formulated to provide the desired viscosity and can be easily spread.

Gel formulations according to the present invention can be prepared using gel bases according to U.S. Pat. No. 4,883,660 A to Blackman et al., incorporated by reference herein in its entirety, where glycol solvents, specifically propylene glycol and polyethylene glycols having average molecular weights of from about 200 to about 800, can be gelled through the addition of small quantities of ethoxylated saturated fatty alcohols, i.e., fatty alcohols having chain lengths of from 16 to 22 carbon atoms which are ethoxylated with 2 to 30 moles of ethylene oxide. While the ethoxylated alcohols are known primarily as nonionic surfactants, they also form very elegant anhydrous gels with glycol solvents, producing gel bases for pharmaceutical compositions that have excellent drug solubilizing, penetration and release characteristics because of the high percentage of glycol solvent in the gels. In addition, the ethoxylated alcohols act as penetration enhancers for the base. The term “gel’ is used herein in the general sense of a semi-solid, apparently homogeneous substance that may be elastic and jelly-like (as gelatin) or more or less rigid. The gel bases can act in themselves as vehicles for active pharmaceutical agents suitable for topical administration in order to achieve a local therapeutic effect, topical administration in order to achieve transdermal or transmucosal penetration and a systemic therapeutic effect, or oral administration. Alternatively, the gel bases can be combined with other ingredients, e.g., diluents, opacifiers, penetrants, coupling agents, fragrances, coloring agents, humectants, moisturizers and the like to provide a total pharmaceutical composition. In addition, for purposes of oral administration, the gels can be incorporated into an oral dosage unit such as a conventional gelatin or other hard capsule, hollow tablet or caplet to provide sustained release from the gel of a pharmaceutical agent upon dissolution of the dosage unit in the gastric juices. Other topical, transdermal, transmucosal and oral dosage forms incorporating the gels of the present invention would be readily apparent to those skilled in the medical and pharmaceutical arts.

The gel bases for pharmaceutical compositions comprise (a) from about 0.5 to about 10.0% by weight CH₃(CH₂)₂₁(OCH₂CH₂)ₙOH and from about 90 to about 99.5% of a glycol solvent, or (b) from about 2.5 to about 10.0% by weight CH₃(CH₂)ₓ(OCH₂CH₂)ₙOH and from about 90 to about 97.5% of a glycol solvent, wherein n is an integer from 2 to 30 and x is an integer from 15 to 20, the bases being substantially free of any added gelling agents. Preferred concentration ranges (by weight) for the components of the gels are from about 0.5 to about 2.5% of ethoxylated behenyl (C₂₂) alcohol and from about 97.5 to about 99.5% of a glycol solvent, or from about 2.5 to about 5% of a C₁₆-C₂₁ ethoxylated alcohol and from about 95 to about 97.5% of a glycol solvent. The preferred range for moles of ethoxylation for all the C₆-C₂₂ alcohols is from 2 to 10. Preferred glycol solvents include propylene glycol and polyethylene glycol having an average molecular weight of about 400. Within the range of C₁₆-C₂₁ ethoxylated alcohols, those alcohols with the highest molecular weight prior to ethoxylation (e.g., behenyl alcohol) and the lowest degree of ethoxylation (e.g., 2 to 5 moles) formed gels with glycols readily even in concentrations as low as 0.5-1.0%. Hence, the most preferred ethoxylated alcohol surfactants for use where low concentration of surfactant in the gel base is desired, for example to minimize irritation to the mucous membranes, are behenyl alcohol ethoxylates (2 to 5 moles of ethoxylation). In addition, these preferred surfactants are highly lipophilic and cause better adhesion of the gel to the phospholipid layers of the mucosa in the nasal and oral cavities. Pharmaceutical agents suitable for incorporation into the gel bases or into pharmaceutical compositions incorporating those gel bases include, but are not limited to, analgesics, decongestants, bronchodilators and other antiasthmatic agents, beta blockers, antihistamines, anesthetics, antifungals, antinauseants, antiemetics, antibacterial agents, antifungal agents, corticosteroids and anticonvulsants. The concentration of the active ingredient in the gel base is, of course, dependent on the identity of the active agent, the condition and patient being treated and the potency desired. For purely topical treatment, for example, treatment of a skin area, compositions with excellent release and penetration characteristics can be formed utilizing the gel bases of the present invention and antibacterials, antifungals, local anesthetics, corticosteroids and similar agents, particularly those which are highly soluble in glycol solvents, and which are suitable for use with anhydrous vehicles. Compositions intended for systemic administration via the mucosa, e.g., intranasally, buccally, or sublingually, can be prepared with the novel gel bases and suitable analgesics, decongestants, bronchodilators, antiasthmatics, antinauseants, anticonvulsants, and other agents where rapid blood level peaks and onset of action are desirable to quickly alleviate the symptoms of the disorder being treated. By virtue of the high glycol solvent content and low surfactant content in the novel gel vehicles, rapid percutaneous and transmucosal absorption are facilitated, and therapeutic blood levels of many agents in these categories can be quickly achieved. Moreover, even at these low concentrations, the selected surfactants augment percutaneous absorption of the active drug ingredient.

The gel bases may be prepared by any conventional method suitable for combining the ethoxylated alcohol surfactant component with the glycol solvent. By one preferred method, the glycol, for example propylene glycol or a polyethylene glycol having a molecular weight of about 200 to 800, is heated to about 80° C., and the surfactant is then stirred in with the mixture being immediately removed from heat. The mixture is allowed to cool to approximately 60°−65° C., at which point an active pharmaceutical ingredient may be stirred into the gel base until a homogeneous mixture, solution or suspension is achieved. In the case of certain active drug ingredients which, because of their solubility characteristics, form precipitates or grainy aggregates when added to the gel bases according to the foregoing method, a modified method for combining the active ingredient with the gel is utilized. The gel base is formulated as described above and allowed to cool to about 60°−65° C., at which point the active ingredient is added, but the gel is then reheated to 70°−80° C. until the active ingredient is well-dissolved in the gel. The gel is then allowed to cool slowly to avoid precipitation of the active ingredient. This method overcomes solubility problems and enables the formation of elegant gels in the case of most pharmaceutical agents soluble in glycols. When the gel bases are formulated with ethoxylated behenyl (C₂₂) alcohol, they have a remarkable capability for retaining substantial quantities of ethyl or isopropyl alcohol without losing their gel-like consistency. Indeed, gel compositions comprising about 50% by weight of a gel base consisting of ethoxylated behenyl alcohol and propylene or polyethylene glycol, and up to 50% alcohol by weight, have been successfully formulated. These gels with high alcohol content are of great utility in solubilizing active ingredients which are poorly soluble in glycol but highly soluble in alcohol. Moreover, the alcohol contained in the gels exerts a bactericidal and bacteriostatic effect on skin areas to which the gels are applied, and provides a cooling counter-balance to the glycol solvent which may sometimes create a warming sensation when applied to the skin. Gel compositions containing high concentrations of ethyl or isopropyl alcohol may not be suitable, however, for application to the mucosa. The gel bases provide unique advantages as vehicles or components of vehicles for pharmaceutical compositions. They comprise a nonionic surfactant-containing system which provides for stable solutions of a wide variety of many pharmaceutical agents with little reactivity of surfactant with drug. Moreover, the non-ionic character of the surfactant and the high glycol concentration aids in the penetration of the gel bases with even high molecular weight active ingredients through the skin and mucous membranes.

Gel pharmaceutical compositions including the gel bases and active pharmaceutical agents may be administered by a variety of methods, depending on the activity of the pharmaceutical agent or agents incorporated in the gel and the condition being treated. Such methods of administration include squeezing the drug-containing gel directly from a tube or other container onto an affected tissue area and spreading a thin film of the gel over that tissue area for topical treatment; inserting the tip of a tube or other suitable container into the nostrils and administering an effective dosage amount of the gel into the nasal passages for rapid dissolution and transmucosal absorption; similar direct application of the gel for transmucosal absorption on other mucous membrane areas, including buccal and sublingual administration; and oral administration of an effective dosage amount of the gel composition directly or within a soluble, orally ingestible outer shell, such as in capsules, tablets or caplets. Other suitable uses and routes of administration for the subject gel bases and pharmaceutical compositions including the same will be apparent to those skilled in the medical and pharmaceutical arts. Among the therapeutic advantages provided by the gel bases and gel compositions containing the same are more rapid and more complete percutaneous absorption of topically active pharmaceutical agents, such as corticosteroids, in comparison with conventional topically applied bases. It is believed that various advantageous features of the novel gel bases, including their high glycol content, their nonionic and anhydrous character, and the absence of any added or extrinsic gelling agents, promote more rapid absorption of topical agents through the outer skin layers and the mucosa. It has also been discovered that certain drugs conventionally utilized for systemic therapy only in the form of salts or esters, not free bases, can be effectively administered transmucosally in free form when incorporated into the gel bases.

Formulations in ointment can include hydrocarbons (e.g., petrolatum), mineral oils, lanolin, and polymers. The polymers can be polyvinyl alcohols (e.g., glycols or fatty alcohols), Carbopol and cellulose derivatives such as methyl cellulose in a combination to provide the correct characteristics of viscosity.

Other formulations meant to cover wounds and provide therapeutic effect contain foaming agents such as sodium laurate sulfate, delivered through a pressurized system that emit a liquid or semisolid dispersion in a gaseous medium. Other natural foaming agents can be used such as those derived from the soap bark tree, decyl glucoside, cocamidopropyl betaine, coco glucoside, and sodium cocoamphoacetate, as well as other well-known surfactants.

Another delivery method would be through modified release formulations and as defined by the United States Pharmacopeia, designed to provide timed-release of the POH or derivatives via a combination of excipients, via nanoparticles, or microencapsulation. The use of excipients in the proper combination to produce the slow release of the POH or derivatives over a 12-24 hr. period such as hydrogels, cellulose derivatives (e.g., hydroxypropyl methyl cellulose, carbomer, carboxypolymethylene), emulsifiers or polymeric emulsifiers, the use of thickening and gel forming agents, and other agents that can provide adequate drug dispersion and provide thermal stability.

Preparation of nanoparticles can be achieved by any individual skilled in the art that can combine excipients such as synthetic polymers (e.g., polyvinyl alcohol, poly-L-lactic acid, polyethylene glycol, and poly (lactic-co-glycolic acid), and natural polymers (e.g., alginate and chitosan) to fabricate nanoparticles. Additionally, micelles, and phospholipids can be used, as well as available compact lipid nanostructures, and the use of green nanoparticulate systems.

Specific compositions for POH and derivatives are presented below:

A preferred cream formulation includes POH or perillartine as an active ingredient (Active), and octadecanol, propanediol, hexadecanol, oleic acid, and mineral oil, as inactive ingredients. The cream formulation acts as a bactericide, fungicide, and disinfectant.

TABLE 20

| Order | Reagent | % |
|---|---|---|
| 1 | Active | 1.019 |
| 2 | Propanediol | 72.994 |
| 3 | Hexadecanol | 11.273 |
| 4 | Oleic Acid | 0.314 |
| 5 | Mineral Oil | 0.308 |
| 6 | Octadecanol | 14.091 |

| | |
|---|---|
| TOTAL | 100.00% |

A preferred ointment formulation includes POH or perillartine as an active ingredient (Active), and bee's wax, mineral oil, and hexadecanol, as inactive ingredients. The ointment formulation is suitable to treat skin and nail fungal infections.

TABLE 21

| Order | Reagent | % |
|---|---|---|
| 1 | Active | 1.193 |
| 2 | Mineral Oil | 85.179 |
| 3 | Hexadecanol | 1.947 |
| 4 | Bee's Wax | 11.68 |
| | (Cera Alba) | |

| | |
|---|---|
| TOTAL | 100.00% |

A preferred gel formulation includes POH or perillartine as an active ingredient (Active), and hydroxypropyl cellulose, tween 60 (polyoxyethylene sorbitan monostearate), DI water, carbopol 940 (polyacrylic acid), sodium bicarbonate, and isopropanol 70%, as inactive ingredients. The gel formulation is suitable to treat skin and nail fungal infections.

TABLE 22

| Order | Reagent | % |
|---|---|---|
| 1 | Active | 0.5-3.0 |
| 2 | polyoxyethylene | 3.635 |
| | sorbitan | |
| | monostearate | |
| | (Tween 60) | |
| 3 | DI water | QS |
| 4 | polyacrylic acid | 0.908 |
| | (Carbopol 940) | |
| 5 | Sodium | 0.454 |
| | bicarbonate | |
| 6 | Isopropanol | 3.572 |
| | 70% | |
| 7 | Hydroxypropyl | 0.545 |
| | cellulose | |

| | |
|---|---|
| TOTAL | 100.00% |

Additionally, another preferred gel formulation includes perillartine as an active ingredient, and hydroxypropyl methylcellulose, carbomer 940, isopropyl myristate 96%, sodium bicarbonate, polyoxyethylene 20-sorbitan monooleate, isopropyl alcohol 70%, and water, as inactive ingredients. This gel formulation is also suitable to treat skin and nail fungal infections.

TABLE 23

| Order | Reagent | % |
|---|---|---|
| 1 | Perillartine | 0.5-3.0 |
| 2 | polyoxyethylene | 3.635 |
| | sorbitan | |
| | monostearate | |
| | (Tween 60) | |
| 3 | DI water | QS |
| 4 | polyacrylic acid | 0.908 |
| | (Carbopol 940) | |
| 5 | Sodium | 0.454 |
| | bicarbonate | |
| 6 | Isopropanol | 3.572 |
| | 70% | |
| 7 | Hydroxypropyl | 0.545 |
| | cellulose | |

| | |
|---|---|
| TOTAL | 100.00% |

The following two preferred liquid formulations include POH or perillartine as an active ingredient (Active). The first formulation includes sodium xylene sulfonate, sodium lauryl sulfate, sodium capryl sulfonate, polyethylene glycol mono (nonylphenyl) ether, isopropyl alcohol 70% and water, as inactive ingredients. The second formulation includes sodium xylene sulfonate, sodium lauryl sulfate, sodium capryl sulfonate, isopropyl alcohol 70% and water, as inactive ingredients. Preferred liquid formulations are suitable for household, pharmaceutical and hospital cleaning and disinfection. This formulation is also suitable for use as a spray to treat bacterial, as well as fungal and yeast infections.

The table below lists the specific composition of a preferred POH or Perillartine formulation.

TABLE 24

| Order | Reagent | % |
|---|---|---|
| 1 | Active | 1.0 |
| 2 | Sodium Xylene | 2.0 |
| | Sulfonate | |
| 3 | Sodium Lauryl Sulfate | 2.0 |
| 4 | Sodium Capryl | 2.0 |
| | Sulfonate | |
| 5 | Polyethylene glycol | 1.0 |
| | mono (nonylphenyl) | |
| | ether | |
| 6 | Isopropyl Alcohol 70% | 20.0 |
| 7 | Water | QS |

| | |
|---|---|
| TOTAL | 100.0% |

The table below lists the specific composition of a preferred formulation.

TABLE 25

| Order | Reagent | % |
|---|---|---|
| 1 | Active | 1.0 |
| 2 | Sodium Xylene | 2.0 |
| | Sulfonate | |
| 3 | Sodium Lauryl Sulfate | 2.0 |
| 4 | Sodium Capryl | 3.0 |
| | Sulfonate | |
| 5 | Isopropyl Alcohol 70% | 50.0 |
| 6 | Water | QS |

| | |
|---|---|
| TOTAL | 100.0% |

A preferred oily topical formulation includes POH or perillartine as an active ingredient (Active) and mineral oil as inactive ingredients. This type of formulation is suitable for topical use to treat bacterial, as well as fungal and yeast infections.

The table below lists the specific composition of a preferred formulation.

TABLE 26

| Order | Reagent | % |
|---|---|---|
| 1 | Perillartine | 0.5-3.0 |
| 2 | Mineral Oil | QS |

| | |
|---|---|
| TOTAL | 100.0% |

According to another embodiment of the invention, the formulation can also be administered orally to the subject.

A preferred mouthwash formulation includes POH or perillartine as an active ingredient (Active) and other typical inactive ingredients used in mouthwash formulations. This type of formulation is suitable for buccal use to treat bacterial, as well as fungal and yeast infections.

The table below lists the specific composition of a preferred formulation.

TABLE 27

| Order | Reagent | % |
|---|---|---|
| 1 | Perillartine | 0.5-3 |
| 2 | Sorbitol | 5-15 |
| 3 | Polyoxyethylenes | 0.5-0.75 |
| | orbitan monolaurate | |
| | (Tween 20) | |
| 4 | Flavoring agent | 0.25 or as |
| | (Thymol, eucalyptol, | desired |
| | menthol, peppermint, | |
| | others) | |
| 5 | Water | QS |

| | |
|---|---|
| TOTAL | 100.0% |

A preferred lozenge formulation includes POH or perillartine as an active ingredient (Active) and other typical inactive ingredients used in lozenges formulations. This type of formulation is suitable for buccal use to treat bacterial, as well as fungal and yeast infections.

The table below lists the specific composition of a preferred formulation.

TABLE 28

| Order | Reagent | mg |
|---|---|---|
| 1 | Perillartine | 4-6 |
| 2 | Magnesium Stearate | 7.5 |
| 3 | Stearic Acid | 75 |
| 4 | Flavoring Agent | 0.25 or as |
| | (Thymol, eucalyptol, | desired |
| | menthol, peppermint, | |
| | fruit flavors) | |
| 5 | Compressible Sugar | QS |

| | |
|---|---|
| TOTAL | 1500 mg |

### Uses and Applications

Thus, the formulations described are applicable to human or animal skin including wounds generated by cutting the skin during a surgical intervention and maintain disinfection post operation as the skin recovers and prevents further infection that can penetrate the skin layers. Similarly, it can help disinfect the broken or scarred skin from dermatophytes and other pathogenic microorganisms. It can be used to treat scratches and bruises from infection in patients of all ages, including diabetic and elderly patients. These scars can also occur after traumatic events such as those treated at emergency rooms and help trauma centers disinfect wounds immediately while triaging incoming patients which may include skin abrasions, bullet, or stab wounds, broken and open skin wounds, and others. The trauma centers would include hospitals and ambulatory or urgent care units, as well as military posts and hospitals to treat wounded soldiers. Cases where wounds can be infected by microorganisms like Acinetobacter baumannii which have caused death or loss of limbs due to infections after post-operatory interventions on the field hospitals during the Gulf War.

Additionally, the formulations can be used to disinfect skin burns at different levels. For example, there are three (3) types of burn wounds classified as first which is superficial, second also recognized as partial thickness, and the third which as full thickness may destroy the epidermis and dermis. This invention can be used in disinfecting the burn wounds caused by thermal, radiation, chemical or electrical burns.

The applications cover the disinfection of intact skin as well as skin with abrasions, scars, burns, scratches, cuts, deep wounds and in general any condition in which the skin is broken and exposed and for which the formulations will assist in maintaining the recovering human or animal free from potential infections during as well as preventing the creation of biofilms. They also include disinfection of the described conditions when they are near nails or on the nail bed itself, as well as disinfect nails from conditions that can cause onychomycoses.

Although the invention has been described with reference to specific preferred applications and formulations, those skilled in the art will appreciate that many modifications and variations to such applications and formulations may be made without departing from the teachings of the invention. All such modifications and variations are intended to be encompassed within the scope of the following claims.

1. A method of reducing pathogenic microorganisms on a subject, the method comprising: administering to said subject in need thereof an effective amount of a formulation comprising at least one monoterpene as an active ingredient selected from the group consisting of Perillyl alcohol, Perillaldehyde, Perillyl ester, Perillyl Imine, Perillartine, and combinations thereof, in a concentration between 0.1% to 3%, wherein said formulation remains active in the presence of blood, inorganic material, organic material, and bodily fluids.

2. The method according to claim 1, wherein said formulation remains active at least for 24 hours.

3. The method according to claim 1, wherein said formulation is administered topically on a skin region of said subject.

4. The method according to claim 3, wherein said formulation is in the form of a gel, cream, ointment, or foam.

5. The method according to claim 3, wherein said skin region includes a wound.

6. The method according to claim 5, wherein said formulation remains active when the wound is occluded.

7. The method according to claim 1, wherein said pathogenic microorganisms comprise at least one of Gram (−) microorganisms, Gram (+) microorganisms, yeast, or fungi.

8. The method according to claim 7, wherein said Gram (−) microorganisms comprise at least one of Enterobacter spp., Escherichia coli, Enterococcus faecalis, Serratia marcescens, Klebsiella pneumoniae, Haemophilus influenzae, Pseudomonas spp., Pseudomonas aeruginosa; Pseudomonas cepacian, Proteus mirabilis, Porphyromonas gingivalis, or Prevotella intermedia.

9. The method according to claim 7, wherein said Gram (+) microorganisms comprise at least one of
Acinetobacter spp., Acinetobacter baumannii, Staphylococcus spp., Bacillus subtilis, Bacillus cereus, Methicillin Resistant Staphylococcus aureus (MRSA), Staphylococcus aureus, Staphylococcus epidermidis, Streptococcus pyogenes, Streptococcus pneumoniae, or Micrococcus yunnanensis.

10. The method according to claim 7, wherein said fungi comprise at least one of Aspergillus spp., Trichophyton rubrum, Aspergillus spp., A. brasiliensis, A. flavus, A. fumigatus, T. mentagrophytes, T. tonsurans, T. verrucosum, or E. floccosum.

11. The method according to claim 7, wherein said yeast comprises at least one of Candida spp., Candida albicans, Candida glabrata, Candida parapsilosis, Candida auris, Candida tropicalis, or Issatchenkia orientalis.

12. The method according to claim 1, wherein said formulation further comprises an anesthetic agent as an inactive ingredient.

13. The method according to claim 12, wherein said anesthetic agent comprises lidocaine, pramoxine, phenol, prilocaine, benzocaine, dibucaine, bupivacaine, proparacaine, epinephrine, capsaicin, menthol, methyl salicylate, tetracaine, camphor, cocaine, dyclonine, or any combination thereof.

14. The method according to claim 1, wherein said formulation further comprises a disinfectant alcohol as an inactive ingredient.

15. The method according to claim 14, wherein said disinfectant alcohol comprises Isopropyl alcohol (IPA), ethanol, or n-propanol.

16. The method according to claim 12, wherein said formulation further comprises a disinfectant alcohol as an inactive ingredient.

17. The method according to claim 16, wherein said disinfectant alcohol comprises Isopropyl alcohol (IPA), ethanol, or n-propanol.

18. The method according to claim 1, wherein said skin region of the subject has at least one of an abrasion, a scar, a burn, a scratch, or a cut.

19. The method according to claim 1, wherein said skin region is at least one of near a nail of the subject, or on a nail bed of the subject.

20. The method according to claim 1, wherein said formulation comprises Perillyl alcohol as the active monoterpene, and N-methyl pyrrolidone (NMP), Polyethylene glycol (15)-hydroxystearate, Polyethylene-polypropylene glycol and water as inactive ingredients.

21. The method according to claim 1, wherein said formulation comprises Perillyl alcohol as the active monoterpene, and Hydroxypropyl-β-Cyclodextrin (HPbCD), Polyvinylpyrrolidone (PVP) and water as inactive ingredients.

22. The method according to claim 14, wherein said formulation comprises Perillyl alcohol as the active monoterpene, and Isopropyl alcohol, polyacrylic acid, and Isopropyl myristate in water as inactive ingredients.

23. The method according to claim 1, wherein said formulation comprises Perillyl alcohol or perillartine as the active monoterpene, and octadecanol, propanediol, hexadecanol, oleic acid, and mineral oil as inactive ingredients.

24. The method according to claim 1, wherein said formulation comprises Perillyl alcohol or perillartine as the active monoterpene, and bee's wax, mineral oil, and hexadecanol as inactive ingredients.

25. The method according to claim 1, wherein said formulation comprises Perillyl alcohol or perillartine as the active monoterpene, and hydroxypropyl cellulose, polyoxyethylene sorbitan monostearate, DI water, polyacrylic acid., sodium bicarbonate and Isopropyl alcohol as inactive ingredients.

26. The method according to claim 1, wherein said formulation comprises perillartine as the active monoterpene, and hydroxypropyl methylcellulose, polyacrylic acid, isopropyl myristate 96%, sodium bicarbonate, polyoxyethylene 20-sorbitan monooloeate, isopropyl alcohol, and water as inactive ingredients.

27. The method according to claim 1, wherein said formulation comprises Perillyl alcohol or perillartine as the active monoterpene, and sodium xylene sulfonate, sodium lauryl sulfate, sodium capryl sulfonate, polyethylene glycol mono (nonylphenyl) ether, isopropyl alcohol and water as inactive ingredients.

28. The method according to claim 1, wherein said formulation comprises Perillyl alcohol or perillartine as the active monoterpene, and sodium xylene sulfonate, sodium lauryl sulfate, sodium capryl sulfonate, isopropyl alcohol and water as inactive ingredients.

29. The method according to claim 1, wherein said formulation comprises Perillyl alcohol or perillartine as the active monoterpene, and mineral as inactive ingredient.

30. The method according to claim 1, wherein said formulation is administered orally to said subject.

31. The method according to claim 30, wherein said formulation comprises Perillyl alcohol or perillartine as the active monoterpene, and sorbitol, Polyoxyethylene sorbitan monostearate, a flavoring agent, and water as inactive ingredients.

32. The method according to claim 30, wherein said formulation comprises Perillyl alcohol or perillartine as the active monoterpene, and Magnesium Stearate, Stearic Acid, and a flavoring agent as inactive ingredients.
